(19) United States
(12) Patent Application Publication
(10) Pub. No.: US 2007/0129410 A1
(43) Pub. Date: Jun. 7, 2007

(54) Octahydrophenanthrene hydrazinde derivatives useful as glucocorticoid receptor modulators

(75) Inventors: Ralph P. Robinson, Gales Ferry, CT (US); Edward F. Kleinman, Pawcatuck, CT (US); Henry Cheng, San Diego, CA (US)

Correspondence Address: PHARMACIA & UPJOHN, 7000 Portage Road, KZO-300-104, KALAMAZOO, MI, 49001, US

(73) Assignee: Pfizer Inc.

(21) Appl. No.: 10/579,354

(22) PCT Filed: Nov. 8, 2004
(86) PCT No.: PCT/IB04/03671

§ 371 Date: Sep. 7, 2006

Related U.S. Application Data
(60) Provisional application No. 60/519,937, filed on Nov. 13, 2003.

(51) Int. Cl. A61K 31/44; C07D 211/68

(52) U.S. Cl. 514/352; 546/285

(57) ABSTRACT

The present invention relates to compounds of the formula (I) their isomer, prodrugs of these compounds or isomers, or a pharmaceutically acceptable salts of these compounds, isomers or prodrugs; wherein R₁, R₂, R₃, R₄, R₅, R₆, R₇, R₈, R₉, and X are defined as in the specification, to pharmaceutical compositions containing them and methods of using these compounds, their isomer, prodrugs of these compounds or isomers, or a pharmaceutically acceptable salts of these compounds, isomers or prodrugs to treat obesity, diabetes, anxiety, or inflammatory diseases or for modulating a process mediated by GR in mammals.

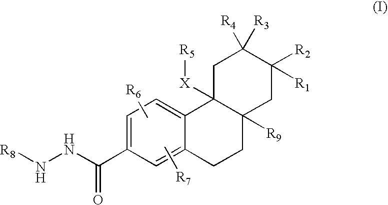

### FIELD OF THE INVENTION

The present invention relates to hydrazide derivatives, methods of preparing these hydrazide derivatives, pharmaceutical compositions containing hydrazide derivatives and methods of using hydrazide derivatives as glucocorticoid receptor modulators and to treat diseases, such as obesity, diabetes, inflammation and others as described below, in mammals.

### BACKGROUND OF THE INVENTION

The glucocorticoid receptor (GR) specifically interacts with DNA and/or protein(s) and regulates their transcription. For example, the GR interacts with the transcription factors, API and NFκ-B to inhibit API- and NF-κ-B-mediated transcription. The inhibition of such API- and NFκ-B-mediated transcription is believed to alleviate inflammatory activity of endogenously administered glucocorticoids.

The activity of the GR can be controlled using GR modulators, such as GR agonists and GR antagonists. Cortisol, corticosterone, dexamethasone, prednisone and prednisilone have been known to be GR agonists. RU486 has been known to be a non-selective GR antagonist. Examples of additional GR modulators are disclosed in U.S. Pat. No. 3,683,091 (phenanthrene compounds); Japanese Patent Application, Publication No. 45014056 (1,2,3,4,9,10,11α,12-octahydro-7-methoxy-12β-butylphenanthren-2β-ol); Japanese Patent Application, Publication No. 6-263688 (phenanthrene derivatives); International Patent Application Publication No. WO 95/10266 (phenanthrene derivatives); Japanese Patent Application, Publication No. 45-36500 (optically active phenanthrene derivatives); European Patent Application, Publication No. 0 188 396 (substituted steroid compounds); C. F. Bigge et al., J. Med. Chem. 1993, 36, 1977-1995 (octahydrophenanthrenamines and certain of their heterocyclic analogues); P. R. Kaniial etat., J. Org. Chem. 1985, 50, 857-863 (complex diterpenoids); G. Sinha et al., J. Chem. Soc., Perkin Trans. I (1983), (10), 2519-2528 (isomeric bridged diketones cis3,4,4a,9,10,10a-hexahydro-1,4a-ethanophenanthren-2(1H), 12-dione and trans3,4,4a,9,10,10a-hexahydro-3,4a-ethanophenanthren-2(1H), 12-dione); U. R. Ghatak, M. Sarkar and S. K. Patra, Tetrahedron Letters No. 32, pp. 2929-2931, 1978 (polycyclic bridged-ring intermediates useful in preparing some complex diterpenoids); P. N. Chakrabortty et al., Indian J. Chem. (1974), 12(9), 948-55 (1α-methyl-1β,4aβ-dicarboxy-1,2,3,4,4a,9,10,10a,β-octahydro- phenanthrene); E. Fujita et al., J. Chem. Soc., Perkin Trans. I (1974), (1), 165-77 (enmein); H. Sdassi et al., Synthetic Communications, 25(17), 2569-2573 (1995) ((R-(+)4a-cyanomethyl-6methoxy-3,4,9,10-tetrahydrophenanthreri-2-one); T. Ibuka et al., Yakugaku Zasshi (1967), 87(8), 1014-17 (alkaloids of menispermaceous plants); Japanese Patent No. 09052899 (diterpene or triterpene derivatives); U.S. Pat. No. 5,696,127 (nonsteroidal compounds, such as 5H-chromeno[3,4-flquinolines); U.S. Pat. No. 5,767,113 (synthetic steroid compounds); Published European Patent Application 0 683 172 (11,21-bisphenyl-19-norpregnane derivatives); D. Bonnet-Delpon et al., Tetrahedron (1996), 52(1), 59-70 (CF₃-substituted alkenes as good partners in Diels-Alder reactions with Danishefsky's diene and in 1,3-dipolar cycloadditions with certain nitrones and non-stabilized azomethine ylides); International Patent Application Publication No. WO 98/26783 (steroid compounds); International Patent Application Publication No. WO 98/27986, (methods for treating non-insulin dependent Diabetes Mellitus or Type II Diabetes, by administering a combination of treatment agents exhibiting GR type I agonist activity and GR type II antagonist activity); International Patent Application Publication No. WO 98/31702 (16-hydroxy-11-(substituted phenyl)-estra4,9-diene derivatives); Published European Patent Application 0 903 146 (steroid 21-hydroxy-6,19-oxidoprogesterone (21OH-60P)); J. A. Findlay et al, Tetrahedron Letters No.19, pp. 869-872, 1962 (intermediates in the synthesis of diterpene alkaloids) and U.S. Pat. No. 6,380,223 (non-steroidal compounds as GR modulators), all of which are incorporated herein by reference.

### SUMMARY OF THE INVENTION

The present invention relates to compounds of the formula I:

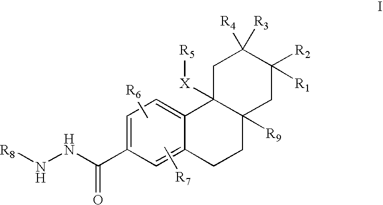

an isomer thereof, a prodrug of said compound or isomer, or a pharmaceutically acceptable salt of said compound, isomer or prodrug; wherein R₁ is a) —H, b) —(C₁-C₆)alkyl-A—(C₀-C₆)alkyl, or —(C₁-C₃)alkyl-A—(C₁-C₃)alkyl-A—(C₀-C₃)alkyl, wherein A for each occurrence is independently S, O, N, OH or NH₂; wherein each carbon atom is optionally substituted with 1 or 2 Rₓ, c) —(C₂-C₁₀)alkenyl optionally substituted with 1 or 2 Rₓ, d) —(C₂-C₁₀)alkynyl, -ethynyl (C₁-C₈)alkoxy or —(C₁-C₄)alkoxy(C₁-C₄)alkylethynyl, wherein each carbon atom is optionally substituted with 0, 1 or 2 Rₓ, e) —CH═C═CH₂, f) —CN, g) —(C₃-C₉)cycloalkyl, h) —Z—(C₆-C₁₀)aryl, i) —Z-het, j) —C(O)O(C₁-C₆)alkyl, k) —O(C₁-C₆)alkyl, l) —Z—S—R₁₂, m) —Z—S(O)—R₁₂, n) —Z—S(O)₂—R₁₂, o) —(C₁-C₈)alkyl, wherein each carbon atom is optionally substituted with 1, 2, or 3 halo, p) —NR₁₂O—(C₁-C₆)alkyl or q) —CH₂ORₓ;

Z for each occurrence is independently a) —(C₀-C₆)alkyl, b) —(C₂-C₆)alkenyl or c) —(C₂-C₆)alkynyl;

Rₓ for each occurrence is independently a) —OH, b) -halo, c) —Z—(C₁-C₈)alkyl, wherein each carbon atom is optionally substituted with 1, 2, or 3 halo, d) —CN, e) —NR₁₂R₁₃, f —(C₃-C₆)cycloalkyl, g) —(C₃-C₆)cycloalkenyl, h) —(C₀-C₃)alkyl-(C₆-C₁₀)aryl, i) -het or j) —N₃; wherein het is a 5-, 6- or 7-membered saturated, partially saturated or unsaturated ring containing from one to three heteroatoms independently selected from the group consisting of nitrogen, oxygen and sulfur; and including any bicyclic group in which any of the above heterocyclic rings is fused to a benzene ring or another heterocycle; and the nitrogen may be in the oxidized state giving the N-oxide form; and optionally substituted with 1, 2 or 3 R_{y};

R_{y} for each occurrence is independently a) -halo, b) —OH, c) —(C₁-C₆)alkyl, d) —(C₂-C₆)alkenyl, e) —(C₂-C₆)alkynyl, f) —O(C₁-C₆)alkyl, g) —O(C₂-C₆)alkenyl, h) —O(C₂-C₆)alkynyl, i) —(C₀-C₆)alkyl-NR₁₂R₁₃, j) —C(O)—NR₁₂R₁₃, k) —Z—SO₂R₁₂, l)—Z—SOR₁₂, m) —Z—SR₁₂, n) —NR₁₂—SO₂R₁₃, o) —NR₁₂—C(O)—R₁₃, p) —NR₁₂—OR₁₃, q) —SO₂—NR₁₂R₁₃, r) —CN, s) —CF₃, t) —C(O)(C₁-C₆)alkyl, u) ═O, or v) —Z—SO₂-phenyl;

R₂, R₃ and R₄ are each independently a) —H, b) -halo, c) —OH, d) —(C₁-C₁₀)alkyl, wherein each carbon atom is optionally substituted with 1, 2 or 3 Rₓ, e) —NR₁₂R₁₃, f) —Z—C(O)O(C₁-C₆)alkyl, g) —Z—C(O)NR₁₂R₁₃, h) (C₁-C₆)alkoxy, i) —Z—O—C(O)—(C₁-C₆)alkyl, j) —Z—O—(C₁-C₃)alkyl-C(O)—NR₁₂R₁₃, k) —Z—O—(C₁-C₃)alkyl-C(O)—O(C₁-C₆)alkyl, l) —O—(C₂-C₆)alkenyl, m) —O—(C₂-C₆)alkynyl, n) —O—Z-het, o) —COOH, p) —C(OH)R₁₂R₁₃ or q) —Z—CN;

R₁₂ and R₁₃ for each occurrence are each independently a) —H, b) —(C₁-C₆)alkyl wherein 1 or 2 carbon atoms, other than the connecting carbon atom, may optionally be replaced with 1 or 2 heteroatoms independently selected from S, O and N and wherein each carbon atom is optionally substituted with 1, 2 or 3 halo, c) —(C₂-C₆)alkenyl optionally substituted with 1, 2 or 3 halo or d) —(C₂-C₆)alkynyl wherein 1 carbon atom, other than the connecting carbon atom and the ethynyl atoms, may optionally be replaced with 1 oxygen atom and wherein each carbon atom is optionally substituted with 1, 2 or 3 halo; or R₁₂ and R₁₃ are taken together with N to which they are attached to form het; X is a) absent, b) —CH₂—, c) —CH(OH)— or d) —C(O)—;

R₅ is a) —H, b) —Z—CF₃, c) —(C₁-C₆)alkyl, d) —(C₂-C₆)alkenyl, e) —(C₂-C₆)alkynyl, f) —(C₆-C₁₀)aryl, g) —CHO, h) —CH═N—OR₁₂, i) —Z—C(O)OR₁₂, j) —Z—C(O)—NR₁₂R₁₃, k) —Z—C(O)—NR₁₂—Z-het, l) —Z—NR₁₂R₁₃, m) —Z—NR₁₂het, n) —Z-het, o) —Z—O-het, p) —Z—(C₆-C₁₀)aryl, q) —Z—O—(C₆-C₁₀)aryl, r) —CHOH—(C₆-C₁₀)aryl or s) —C(O)—(C₆-C₁₀)aryl wherein said (C₆-C₁₀)aryl is optionally substituted with 1 or 2 of the following: —Z—OH, —Z—NR₁₂R₁₃, —Z—NR₁₂-het, —C(O)NR₁₂R₁₃, —C(O)O(C₁-C₆)alkyl, —C(O)OH, —C(O)-het, —NR₁₂—C(O)—(C₁-C₆)alkyl, —NR₁₂—C(O)—(C₂-C₆)alkenyl, —NR₁₂—C(O)—(C₂-C₆)alkynyl, —NR₁₂—C(O)—Z-het, —CN, —Z-het, —O—(C₁-C₃)alkyl-C(O)—NR₁₂R₁₃, —O—(C₁-C₃)alkyl-C(O)O(C₁-C₆)alkyl, —NR₁₂—Z—C(O)O(C₁-C₆)alkyl, —N(Z—C(O)O(C₁-C₆)alkyl)₂, —NR₁₂—Z—C(O)-NR₁₂R₁₃, —Z—NR₁₂—SO—R₁₃, —NR₁₂—SO₂-het, —C(O)H, —Z—NR₁₂—Z—O(C₁-C₆)alkyl, —Z—NR₁₂—Z—NR₁₂R₁₃, —Z—NR₁₂-(C₃-C₆)cycloalkyl, —Z—N(Z—O(C₁-C₆)alkyl)₂, —SO₂R₁₂, —SOR₁₂, —SR₁₂, —SO₂NR₁₂R₁₃, —O—C(O)—(C₁-C₄)alkyl, —O—SO₂—(C₁-C₄)alkyl, -halo or —CF₃; R₆ and R₉ are each independently a) —H, b) -halo, c) (C₁-C₆)alkyl substituted with 0 to 3 halo, d) —(C₂-C₆)alkenyl substituted with 0 to 3 halo, e) —(C₂-C₆)alkynyl optionally substituted with 1, 2 or 3 halo, f) —CN, g) —(C₃-C₆)cycloalkyl, h) —(C₃-C₆)cycloalkenyl, i) —OH, j) —O—(C₁-C₆)alkyl, k) —O—(C₁-C₆)alkenyl, l) —O—(C₁-C₆)alkynyl, m) —NR₁₂R₁₃, n) —C(O)OR₁₂ or o) —C(O)NR₁₂R₁₃;

R₇ is a) —H, b) —(C₁-C₁₀)alkyl optionally substituted with 1, 2 or 3 substituents independently selected from -halo, —OH and —N₃, c) —(C₂-C₁₀)alkenyl optionally substituted with 1, 2 or 3 substituents independently selected from -halo, —OH and —N₃, d) —(C₂-C₁₀)alkynyl optionally substituted with 1, 2 or 3 substituents independently selected from -halo, —OH and —N₃, e) -halo, —O—Z—CN, g) —OH, h) —Z-het, i) —Z—NR₁₂R₁₃, j) —Z—C(O)-het, k) —Z—C(O)—(C₁-C₆)alkyl, l) —Z—C(O)—NR₁₂R₁₃, m) —Z—C(O)—NR₁₂—Z—CN, n) —Z—C(O)—NR₁₂—Z-het, o) —Z—C(O)—NR₁₂—Z—(C₆-C₁₀)aryl, p) —Z—C(O)—NR₁₂—Z—NR₁₂R₁₃, q) —Z—C(O)—NR₁₂—Z—O(C₁-C₆)alkyl, r) —(C₀-C₆)alkyl-C(O)OH, s) —Z—C(O)O(C₁-C₆)alkyl, t) —Z—O—(C₀-C₆)alkyl-het, u) —Z—O—(C₀-C₆)alkyl-(C₆-C₁₀)aryl, v) —Z—O—(C₁-C₆)alkyl optionally substituted with 1 or 2 R_{y}, w) —Z—O—(C₁-C₆)alkyl-CH(O), x) —Z—O—(C₁-C₆)alkyl—NR₁₂-het, y) —Z—O—Z-het-Z-het, z) —Z—O—Z-het-Z—NR₁₂R₁₃, a1) —Z—O—Z-het-C(O)-het, b1) —Z—O—Z—C(O)-het, c1) —Z—O—Z—C(O)-het-het, d1) —Z—O—Z—C(O)—(C₁-C₆)alkyl, e1) —Z—O—Z—C(S)—NR₁₂R₁₃, f1) —Z—O—Z—C(O)—NR₁₂R₁₃, g1) —Z—O—Z—(C₁-C₃)alkyl-C(O)—NR₁₂R₁₃, h1) —Z—O—Z—C(O)—O(C₁-C₆)alkyl, i1) —Z—O—Z—C(O)OH, j1) —Z—O—Z—C(O)—NR₁₂—O(C₁-C₆)alkyl, k1) —Z—O—Z—C(O)—NR₁₂—OH, l1) —Z—O—Z—C(O)—NR₁₂—Z—NR₁₂R₁₃, m1) —Z—O—Z—C(O)—NR₁₂—Z-het, n1) —Z—O—Z—C(O)NR₁₂—SO₂—(C₁-C₆)alkyl, o1) —Z—O—Z—C(═NR₁₂)(NR₁₂R₁₃), p1) —Z—O—Z—C(═NOR₁₂)(NR₁₂R₁₃), q1) —Z—NR₁₂—C(O)—O—Z—NR₁₂R₁₃, r1) —Z—S—C(O)—NR₁₂R₁₃, s1) —Z—O—SO₂—(C₁-C₆)alkyl, t1) —Z—O—SO₂—(C₆-C₁₀)aryl, u1) —Z—O—SO₂—NR₁₂R₁₃, v1) —Z—O—SO₂—CF₃, w1) —Z—NR₁₂C(O)OR₁₃ or x1) —Z—NR₁₂C(O)R₁₃;

R₈ is het.

The compounds of the invention also exist in different tautomeric forms. This invention relates to all tautomers of formula I. The compounds of this invention may contain olefin-like double bonds. When such bonds are present, the compounds of the invention exist as cis and trans configurations and as mixtures thereof.

All stereoisomers (e.g., cis and trans isomers) and all optical isomers of compounds of the formula I (e.g., R and S enantiomers), as well as racemic, diastereomeric and other mixtures of such isomers are within the scope of the present invention.

The compounds of the present invention are named according to the IUPAC or CAS nomenclature system.

In one way of naming the compounds of the present invention, the carbon atoms in the ring of the compounds of the present invention may be numbered as shown in the following simplified structure:

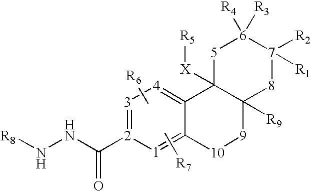

Alternatively, another way of naming the compounds of the present invention, the carbon atoms in the ring may be numbered as shown in the following simplified structure:

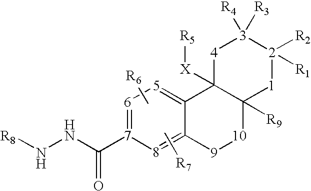

Unless otherwise indicated, the carbon atom content of various hydrocarbon-containing moieties is indicated by a prefix designating the minimum and maximum number of carbon atoms in the moiety, i.e., the prefix Cᵢ-Cⱼ indicates a moiety of the integer “i” to the integer “j” carbon atoms, inclusive. Thus, for example, C₁-C₃ alkyl refers to alkyl of one to three carbon atoms, inclusive, or methyl, ethyl, propyl and isopropyl, and all isomeric forms and straight and branched forms thereof.

Examples of alkyl of one to nine carbon atoms, inclusive, are methyl, ethyl, propyl, butyl, pentyl, hexyl, heptyl, octyl, and nonyl, and all isomeric forms and straight and branched thereof.

Examples of alkenyl of two to five carbon atoms, inclusive, are ethenyl, propenyl, butenyl, pentenyl, and all isomeric forms and straight and branched forms thereof.

Examples of alkynyl of two to five carbon atoms, inclusive, are ethynyl, propynyl, butynyl, pentynyl and all isomeric forms and straight and branched forms thereof.

As used herein, the terms “cycloalkyl, cycloalkenyl and cycloalkynyl” refer to, but are not limited to, cyclic forms of alkyl, alkenyl and alkynyl, respectively. Exemplary (C₃-C₈)cycloalkyl groups are cyclopropyl, cyclobutyl, cyclopentyl, cyclohexyl, cycloheptyl and cyclooctyl.

As used herein the term “halo” includes chloro, bromo, iodo and fluoro.

As used herein, the term “aryl” refers to an optionally substituted aromatic ring, including polyaromatic rings. Examples of aryl include phenyl, naphthyl and biphenyl. An example of six membered aryl is phenyl.

As used herein, the term “het” refers to an optionally substituted 5-, 6- or 7-membered saturated, partially saturated or unsaturated heterocyclic ring containing from 1 to 3 heteroatoms selected from the group consisting of nitrogen, oxygen and sulfur; and including any bicyclic group in which any of the above heterocyclic rings is fused to a benzene ring or another heterocyclic ring; and the nitrogen atom may be in the oxidized state giving the N-oxide form; and substituted by 0 to 3 independent substituents.

The following paragraphs describe exemplary heterocyclic ring(s) for the generic ring descriptions contained herein.

Exemplary five-membered heterocyclic rings are furyl, thienyl, 2H-pyrrolyl, 3H-pyrrolyl, pyrrolyl, 2-pyrrolinyl, 3pyrroilnyi, pyrrolidinyl, 1,3-dioxolanyl, oxazolyl, thiazolyl, imidazolyl, 2H-imidazolyl, 2-imidazolinyl, imidazolidinyl, pyrazolyl, 2-pyrazolinyl, pyrazolidinyl, isoxazolyl, isothiazolyl, 1,2-dithiolyl, 1,3-dithiolyl, 3H-1,2-oxathiolyl, 1,2,3-oxadizaolyl, 1,2,4-oxadiazolyl, 1,2,5-xadiazolyl, 1,3,4-oxadiazolyl, 1,2,3-triazolyl, 1,2,4-trizaolyl, 1,3,4-thiadiazolyl, 1,2,3,4-oxatriazolyl, 1,2,3,5-oxatrizaolyl, 3H-1,2,3-dioxazolyl, 1,2,4-dioxazolyl, 1,3,2-dioxazolyl, 1,3,4-dioxazolyl, 5H-1,2,5-oxathiazolyl and 1,3-oxathiolyl.

Exemplary six-membered heterocyclic rings are 2H-pyranyl, 4H-pyranyl, pyridinyl, piperidinyl, 1,2-dioxinyl, 1,3-dioxinyl, 1,4-dioxanyl, morpholinyl, 1,4-dithianyl, thiomorpholinyl, pyridazinyl, pyrimidinyl, pyrazinyl, piperazinyl, 1,3,5-triazinyl, 1,2,4-triazinyl, 1,2,3-trizainyl, 1,3,5-trithianyl, 4H-1,2-oxazinyl, 2H-1,3-oxazinyl, 6H-1,3-oxazinyl, 6H-1,2-oxazinyl, 1,4-oxazinyl, 2H-1,2-oxazinyl, 4H-1,4-oxazinyl, 1,2,5-oxathiazinyl, 1,4-oxazinyl, o-isoxazinyl, p-isoxazinyl, 1,2,5-oxathiazinyl, 1,2,6-oxathiazinyl, 1,4,2-oxadiazinyl and 1,3,5,2-oxadiazinyl.

Exemplary seven-membered heterocyclic rings are azepinyl, oxepinyl, thiepinyl and 1,2,4-diazepinyl.

Exemplary eight membered heterocyclic rings are cyclooctyl, cyclooctenyl and cyclooctadienyl.

Exemplary bicyclic rings consisting of combinations of two fused partially saturated, fully saturated or fully unsaturated five or six membered rings, taken independently, optionally having one to four heteroatoms selected independently from nitrogen, sulfur and oxygen are indolizinyl, indolyl, isoindolyl, 3H-indolyl, 1H-isoindolyl, indolinyl, cyclopenta(b)pyridinyl, pyrano(3,4-b)pyrrolyl, benzofuryl, isobenzofuryl, benzo(b)thienyl, benzo(c)thienyl, 1H-indazolyl, indoxazinyl, benzoxazolyl, anthranilyl, benzimidazolyl, benzthiazolyl, purinyl, 4Hquinolizinyl, quinolinyl, isoquinolinyl, cinnolinyl, phthalazinyl, quinazolinyl, quinoxalinyl, 1,8-naphthyridinyl, pteridinyl, indenyl, isoindenyl, naphthyl, tetralinyl, decalinyl, 2H-1-benzopyranyl, pyrido(3,4-b)-pyridinyl, pyrido(3,2-b)-pyridinyl, pyrido(4,3-b)-pyridinyl, 2H-1,3-benzoxazinyl, 2H-1,4-benzoxazinyl, 1H-2,3-benzoxazinyl, 4H-3,1-benzoxazinyl, 2H-1,2-benzoxazinyl and 4H-1,4-benzoxazinyl.

As used herein, the term “heteroaryl” refers to an optionally substituted aromatic ring containing from 1 to 3 heteroatoms selected from the group consistng of nitrogen, oxygen and sulfur; and including any bicyclic group in which any of the above rings is fused to a benzene ring or another heterocyclic ring; and the nitrogen atom may be in the oxidized state giving the N-oxide form; and substituted by 0 to 3 independent substtuents.

As used herein the term “mammals” is meant to refer to all mammals, including, for example, primates such as humans and monkeys. Examples of other mammals included herein are rabbits, dogs, cats, cattle, goats, sheep and horses.

As used herein, the term “treating”, “treat” or “treatment” as used herein includes preventative (e.g., prophylactic) and palliative treatment.

By “pharmaceutically acceptable” it is meant the carrier, vehicle, diluent, excipient must be compatible with the other ingredients of the formulation, and not deleterious to the recipient thereof.

As used herein, the term “prodrug” refers to compounds that are drug precursors which following administration, release the drug in vivo via some chemical or physiological process (e.g., a prodrug on being brought to the physiological pH or through enzyme action is converted to the desired drug form). Exemplary prodrugs upon cleavage release the corresponding free acid, and such hydrolyzable ester-forming residues of the Formula I compounds include but are not limited to those having a carboxyl moiety wherein the free hydrogen is replaced by (C₁-C₄)alkyl, (C₂-C₇)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N-(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl.

Some of the compounds of this invention are acidic and they form salts with pharmaceutically acceptable cations. Some of the compounds of this invention are basic and they form salts with pharmaceutically acceptable anions. All such salts, including di-salts, are within the scope of this invention and they can be prepared by conventional methods, such as by contacting the acidic and basic entities, in either an aqueous, non-aqueous or partially aqueous medium. For example, the mesylate salt is prepared by reacting the free base form of the compound of Formula I with methanesulfonic acid under standard conditions. Likewise, the hydrochloride salt is prepared by reacting the free base form of the compound of Formula I with hydrochloric acid under standard conditions. The salts are recovered either by filtration, by precipitation with a non-solvent followed by filtration, by evaporation of the solvent, or, in the case of aqueous solutions, by lyophilization, as appropriate.

In addition, when the compounds and prodrugs of the present invention form hydrates or solvates, they are also within the scope of the present invention.

The compounds and prodrugs of the present invention also includes racemates, stereoisomers and mixtures of these compounds, including isotopically labeled and radiolabeled compounds. Such isomers can be isolated by standard resolution techniques, including fractional crystallization and chiral column chromatography.

For instance, the compounds of the present invention have asymmetric carbon atoms and are therefore enantiomers or diastereomers. Diasteromeric mixtures can be separated into their individual diastereomers on the basis of their physical/chemical differences by methods known in the art, for example, by chromatography and/or fractional crystallization. Enantiomers can be separated by converting the enantiomeric mixture into a diasteromeric mixture by reaction with an appropriate optically active compound (e.g., alcohol), separating the diastereomers and converting (e.g., hydrolyzing) the individual diastereomers to the corresponding pure enantiomers. All such isomers, including diastereomers, enantiomers and mixtures thereof are considered as part of this invention.

The following configurations of the compounds of the present invention (as represented by simplified structures) are preferred, with the first configuration being more preferred:

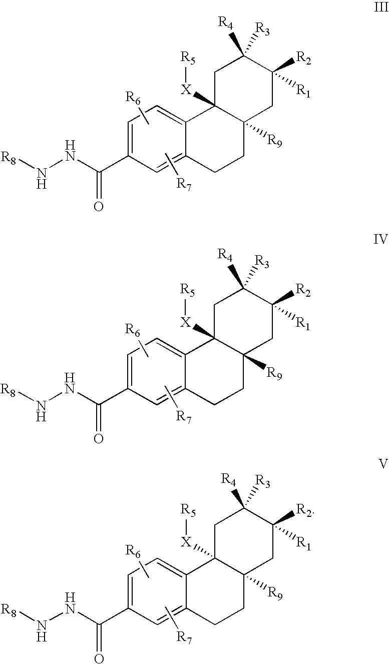

Also, the compounds and prodrugs of the present invention can exist in several tautomeric forms, including the enol form, the keto form and mixtures thereof. All such tautomeric forms are included within the scope of the present invention.

An embodiment of the present invention includes compounds of formula I wherein het in all instances is a heteroaryl having five to seven members.

Another embodiment of the present invention includes compounds of formula wherein R₁ is a) —H, b) —(C₁-C₁₀)alkyl, wherein each carbon atom is optionally substituted with 1, 2 or 3 Rₓ, c) —(C₂-C₁₀)alkenyl optionally substituted with 1 or 2 Rₓ, d) —(C₂C₁₀)alkynyl, wherein each carbon atom is optionally substituted with 1 or 2 Rₓ, e) —(C₃-C₆)cycloalkyl, f) —Z—(C₆-C₁₀)aryl, or g) —Z-heteroaryl having five to seven members;

wherein Rₓ for each occurrence is independently —OH, -halo, and —Z—CF₃;

wherein R₂ is a) —H, b) -halo, c) —OH, d) —(C₁-C₆)alkyl optionally substituted with —OH, e) —Z-heteroaryl having five to seven members, f) —COOH, g) —(C₁-C₁₀)alkyl, wherein each carbon atom is optionally substituted with 1, 2 or 3 Rₓ.

Another embodiment of the present invention includes compounds of formula I wherein R₃ and R₄ are each independently a) —H, b) -halo, c) —OH, d) —(C₁-C₆)alkyl optionally substituted with —OH, e) —Z-heteroaryl having five to seven members, f) —COOH, g)—(C₁-C₁₀)alkyl, wherein each carbon atom is optionally substituted with 1, 2 or 3 Rₓ; wherein Rₓ for each occurrence is independently —OH, -halo, and —Z—CF₃.

Another embodiment of the present invention includes compounds of formula I wherein R₅ is a) —H, b) —Z—CF₃, c) —(C₁-C₆)alkyl, d) —(C₂-C₆)alkenyl, e) —(C₂-C₆)alkynyl, f) —(C₆-C₁₀)aryl, g) —CHO, h) —CH=N—OR₁₂, i) —Z—C(O)OR₁₂, j) —Z—C(O)—NR₁₂R₁₃, k) —Z—C(O)NR₁₂—Z-heteroaryl having five to seven members, l) —Z—NR₁₂R₁₃, m) —Z—NR₁₂-heteroaryl having five to seven members, n) —Z-heteroaryl having five to seven members, o) —Z—O-heteroaryl having five to seven members.

Another embodiment of the present invention includes compounds of formula I wherein R₆ and R₉ are each independently a) —H, b) -halo, c) (C₁-C₆)alkyl optionally substituted with 1, 2 or 3 halo, d) —(C₂-C₆)alkenyl optionally substituted with 1, 2 or 3 halo, e) —(C₂-C₆)alkynyl optionally substituted with 1, 2 or 3 halo, f) —CN, g) —(C₃-C₆)cycloalkyl, h) —(C₃-C₆)cycloalkenyl, i) —OH, j) —O—(C-C₆)alkyl, k) —O—(C₁-C₆)alkenyl, I) —O—(C₁-C₆)alkynyl, m) —NR₁₂R₁₃, n) —C(O)OR₁₂ or o) —C(O)NR₁₂R₁₃.

Another embodiment of the present invention includes compounds of formula I wherein R₇ is a) —H, b) —(C₁-C₁₀)alkyl optionally substituted with 1, 2 or 3 substituents independently selected from -halo, —OH and —N₃, c) —(C₂-C₁₀)alkenyl optionally substituted with 1, 2 or 3 substituents independently selected from -halo, —OH and —N₃, d) —(C₂-C₁₀)alkynyl optionally substituted with 1, 2 or 3 substituents independently selected from -halo, —OH and —N₃, e) -halo, f) —Z—CN, g) —OH, or h) —Z-heteroaryl having five to seven members.

A further embodiment of the present invention includes compounds of formula I wherein R₈ is a 6membered unsaturated ring.

Examples of preferred compounds of formula I are the followings 4b-Ethyl-7-hydroxy-7-trifluoromethyl-4b,5,6,7,8,8a,9,10-octahydro-phenanthrene-2-carboxylic acid N′-pyridin-2-yl-hydrazide , 4b-Benzyl-7-hydroxy-7-trifluoromethyl-4b,5,6,7,8,8a,9,10-octahydro-phenanthrene-2-carboxylic acid N′-pyridin-2-yl-hydrazide, 4b-Ethyl-6,7-dihydroxy-methyl-7-thiazol-2-yl-4b,5,6,7,8,8a,9,10-octahydro-phenanthrene-2-carboxylic acid N′-pyridin-2-yl-hydrazide and all their isomers

The present invention also relates to pharmaceutical compositions for treating obesity, diabetes, anxiety, or inflammatory diseases or for modulating a process mediated by glucocorticoid receptor in a mammal comprising (1) the compounds of formula I or their isomers, prodrugs of the compounds or isomers, or a pharmaceutically acceptable salts of these compounds, isomer or prodrugs and (2) at least one pharmaceutically acceptable carrier, vehicle, diluent, excipient.

One embodiment of the invention includes methods of treating obesity, diabetes, anxiety, or inflammatory diseases in a mammal comprising administering an effective amount of compounds of formula I, isomers thereof, prodrugs of these compounds or isomers, or pharmaceutically acceptable salts of these compounds, isomers or prodrugs.

Another embodiment of the invention includes methods of treating inflammatory diseases selected from the group consisting of arthritis, asthma, rhinitis and immunomodulation.

The present invention also relates to pharmaceutical compositions comprising (1) compounds of formula I, isomers thereof, prodrugs of these compounds or isomers, or pharmaceutically acceptable salts of these compounds, isomers or prodrugs, (2) a second pharmaceutically active compound, and (3) at least one pharmaceutically acceptable carrier, vehicle, diluent, excipient.

One embodiment of the invention includes compositions having a second pharmaceutically active compound selected from the group consisting of P3 agonist, a thyromimetic agent, an eating behavior-modifying agent, a NPY antagonist, an aldose reductase inhibitor, a glycogen phosphorylase inhibitor, a sorbitol dehydrogenase inhibitor, insulin, troglitazone, sulfonylureas, glipazide, glyburide, chlorpropamide, a glucocorficoid receptor agonist, a cholinomimetic drug, an anti-Parkinson's drug, an antianxialytic drug, an antidepressant drug, or an antipsychotic drug.

Another embodiment of the invention includes compositions for treating anti-Parkinson's drug selected from the group consisting of L-dopa, bromocriptine and selegiline.

Another embodiment of the invention includes compositions wherein the second drug is an anbanxiolytic drug selected from the group consisting of benzodiazepine, valium and librium.

Another embodiment of the invention includes compositions wherein the second drug is an antidepressant drug selected from the group consisting of desipramine, sertraline hydrochloride and fluoxetine hydrochloride.

Another embodiment of the invention includes compositions wherein the second drug is an antpsychotic drug selected from the group consisting of haloperidol and clozapine.

Another embodiment of the invention includes compositions wherein the second drug is a glucocorticoid receptor agonist selected from the group consisting of prednisone, prednylidene, prednisolone, cortisone, dexamethasone and hydrocortisone.

The present invention also relates to processes of preparing compounds of formula I, an isomer thereof, a prodrug of said compound or isomer, or a pharmaceutically acceptable salt of said compound, isomer or prodrug, comprising the step of coupling compound of formula Id with a hydrazine under amide forming conditions:

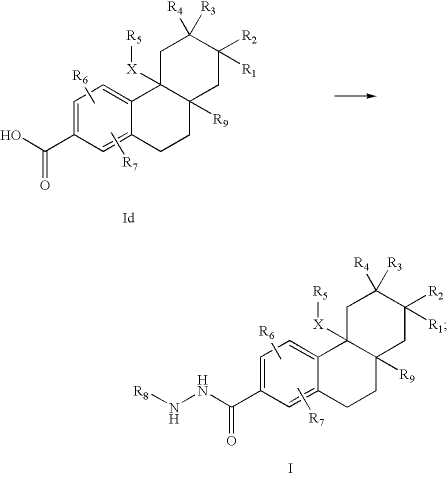

wherein R₁, R₂, R₃, R₄, R₅, R₆, R₇, R₈, R₉ and X are as defined in formula I.

### DETAILED DESCRIPTION OF THE INVENTION

Compounds of formula I of the present invention are prepared as described in the Schemes, Preparations and Examples below, or are prepared by methods analogous thereto, which are readily known and available to one of ordinary skill in light of this disclosure. In each of the Schemes, the R groups (e.g., R₁, R₂, etc.) correspond to those noted in the Summary above. However, it will be understood by those skilled in the art that other functionalities disclosed herein at the indicated positions of compounds of Formula I also comprise potential substituents for the analogous positions on the structures within the Schemes.

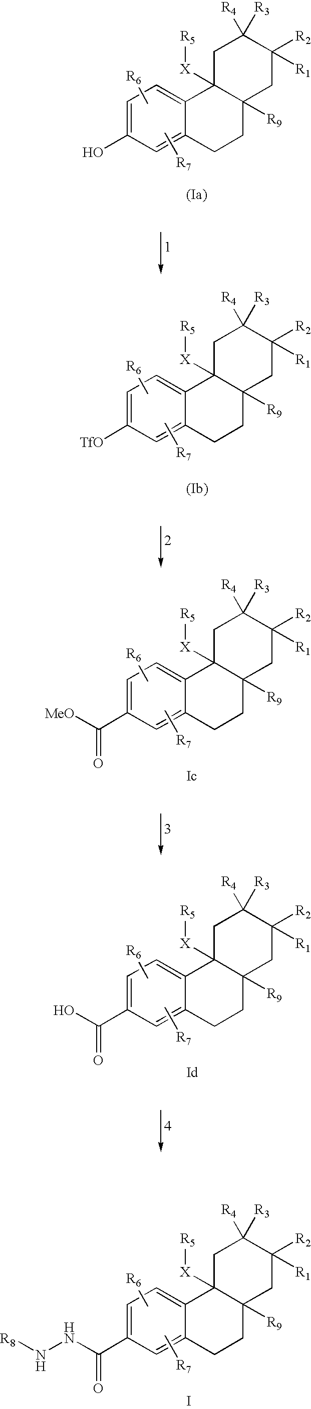

The starting phenol (compound Ia) is dissolved in an organic solvent such as THF, then deprotonated with a base such as NaH. The dissolution and deprotonization of Compound la can be accomplished at room temperature, e.g., from about 5° C. to about 40° C. for a period of time of up to about 4 hours, preferably from about 30 minutes to 1 hour.

Compound Ia is then reacted with N-phenyltrifluoromethane sulfonimide at room temperature for up to 100 hours, preferably from about 48 to about 96 hours, to produce its triflate derivative (compound Ib).

The triflate derivative is subjected to a catalyzed reaction with a catalyst such as palladium (II) acetate in the presence of bisdiphenylphosphenopropane, CO and MeOH to produce the methyl ester (compound Ic).

The methyl ester is then hydralyzed using standard conditions such as LiOH/MeOH to produce the acid (compound Id).

The acid is then coupled with hydrazine under conditions that promotes amide formation to produce compound 1, preferably through EDC/HOBt coupling.

The prodrug can be readily prepared from the inventive compounds using methods known in the art, such as those described by Burger's Medicinal Chemistry and Drug Chemistry, Fifth Ed., Vol. 1, pp. 172-178, 949-982 (1995).

The pharmaceutically acceptable acid addition salts of compounds of the formula I can be prepared by reacting the aforementioned base compounds of this invention with a non-toxic acid, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3-naphthoate)]salts.

The pharmaceutically acceptable base addition salts of compounds of the formula I can be prepared by reacting the aforementioned acid compounds of this invention with a non-toxic base, which contains a pharmacologically acceptable cation such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other pharmaceutically acceptable organic amines.

The racemates of the compounds of Formula I can be separated into their individual isomers mechanically as by chromatography using a chiral absorbent. Alternatively, the individual isomers can be prepared in chiral form or separated chemically from a mixture by forming salts with a chiral acid, such as the individual enantiomers of 10-camphorsulfonic acid, camphoric acid, α-bromocamphoric acid, methoxyacetic acid, tartaric acid, diacetyltartaric acid, malic acid, pyrrolidone-5-carboxylic acid, and the like, and then freeing one or both of the resolved bases, optionally repeating the process, so as obtain either or both substantially free of the other; i.e., in a form having an optical purity of>95%.

The pharmaceutical compositions and compounds, isomers, prodrugs and pharmaceutically acceptable salts thereof of the present invention will generally be administered in the form of a dosage unit (e.g., tablet, capsule, etc.) at a therapeutically effective amount of such compound, prodrug or salt thereof from about 0.1 μg/kg of body weight to about 500 mg/kg of body weight, more particularly from about 1 μg/kg to about 250 mg/kg, and most particularly from about 2 μg/kg to about 100 mg/kg. More preferably, a compound of the present invention will be administered at an amount of about 0.1 mg/kg to about 500 mg/kg of body weight, and most preferably from about 0.1 mg/kg to about 50 mg/kg of body weight. As recognized by those skilled in the art, the particular quantity of pharmaceutical composition according to the present invention administered to a patient will depend upon a number of factors, including, without limitation, the biological activity desired, the condition of the patient, and tolerance for the drug.

Methods of preparing various pharmaceutical compositions with a certain amount of active ingredient are known, or will be apparent in light of this disclosure, to those skilled in this art. For examples of methods of preparing pharmaceutical compositions, see Remington's Pharmaceutical Sciences, Mack Publishing Company, Easter, Pa., 15th Edition (1975), the content of which is hereby incorporated by reference.

One of ordinary skill in the art will appreciate that the compounds of the invention are useful in treating a diverse array of diseases. The GR agonists, partial agonists and antagonists of the present invention can be used to influence the basic, life sustaining systems of the body, including carbohydrate, protein and lipid metabolism, electrolyte and water balance, and the functions of the cardiovascular, kidney, central nervous, immune, skeletal muscle and other organ and tissue systems. In this regard, GR modulators are used for the treatment of diseases associated with an excess or a deficiency of glucocorticoids in the body. As such, they may be used to treat the following: obesity, diabetes, cardiovascular disease, hypertension, Syndrome X, depression, anxiety, glaucoma, human immunodeficiency virus (HIV) or acquired immunodeficiency syndrome (AIDS), neurodegeneration (for example, Alzheimer's and Parkinson's), cognition enhancement, Cushing's Syndrome, Addison's Disease, osteoporosis, frailty, inflammatory diseases (such as osteoarthritis, rheumatoid arthritis, asthma and rhinitis), tests of adrenal function, viral infection, immunodeficiency, immunomodulation, autoimmune diseases, allergies, wound healing, compulsive behavior, multi-drug resistance, addiction, psychosis, anorexia, cachexia, post-traumatic stress syndrome, post-surgical bone fracture, medical catabolism and prevention of muscle frailty.

One of ordinary skill in the art will also appreciate that when using the compounds of the invention in the treatment of a specific disease that the compounds of the invention may be combined with various existing therapeutic agents used for that disease.

For the treatment of obesity, the compounds of the invention may be combined with agents like 3 agonist, thyromimetic agent, eating behavior modifying agent, and NPY antagonist.

The compounds of the invention can also be used in combination with existing therapeutic agents for the treatment of diabetes. Suitable agents to be used in such a combination include aldose reductase inhibitor, glycogen phosphorylase inhibitor, sorbitol dehydrogenase inhibitor, insulin, troglitazone, sulfonylureas, glipazide, glyburide and chlorpropamide.

The compounds of the invention can also be used in combination with other therapeutic agents, which include GR agonists, cholinomimetic drugs, anti-Parkinson's drugs, antianxialytic drugs, antidepressant drugs, and antipsychotic drugs. Examples of GR agonists include prednisone, prednylidene, prednisolone, cortisone, dexamethasone and hydrocortisone. Examples of anti-Parkinson's drugs include L-dopa, bromocriptine and selegiline. Examples of antianxialytic drugs include benzodiazepine, valium and librium. Examples of antidepressant drugs include desipramine, sertraline hydrochloride and fluoxetine hydrochloride. Examples of antipsychotic drug include haloperidol and clozapine.

In combination therapy treatment, both the compounds of this invention and the other drug therapies are administered to mammals (e.g., humans, male or female) by conventional methods. As recognized by those skilled in the art, the therapeutically effective amounts of the compounds of this invention and the other drug therapies to be administered to a patient in combination therapy treatment will depend upon a number of factors, including, without limitation, the biological activity desired, the condition of the patient, and tolerance for the drug.

For example, the second compound of this invention, when administered to a mammal, is dosed at a range between about 0.01 to about 50 mg/kglday body weight, preferably about 0.1 mg/kglday to about 10 mg/kg/day body weight, administered singly or as a divided dose.

As noted above, the compounds, isomers, prodrugs and pharmaceutically acceptable salts of the present invention can be combined in a mixture with a pharmaceutically acceptable carrier, vehicle or diluent to provide pharmaceutical compositions useful for treating the biological conditions or disorders noted herein in mammalian, and more preferably, in human, patients. The particular carrier, vehicle or diluent employed in these pharmaceutical compositions may take a wide variety of forms depending upon the type of administration desired, for example, intravenous, oral, topical, suppository or parenteral. Also, the compounds, isomers, prodrugs and salts thereof of this invention can be administered individually or together in any conventional dosage form, such as an oral, parenteral, rectal or transdermal dosage form.

For oral administration a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols. When aqueous suspensions and/or elixirs are desired for oral administration, the compounds, prodrugs and pharmaceutically acceptable salts thereof of this invention can be combined with various sweetening agents, flavoring agents, coloring agents, emulsifying agents and/or suspending agents, as well as such diluents as water, ethanol, propylene glycol, glycerin and various like combinations thereof.

The activity of the compound I are demonstrated by one or more of the assays described below:

The following is a description of an assay for the identificabon of glucocorticoid receptor antagonists/agonists: SW 1353 human chondrosarcoma cells containing endogenous human glucocorticoid receptors are transfected with a 3×GRE-luciferase plasmid generated by standard procedures and a plasmid conferring neomycin resistance. Novel glucocorticoid responsive cell lines are generated and characterized. One such cell line designated SW 1353 human chondrosarcoma is used for determining the activity of compounds at the glucocorticoid receptor. Cells are maintained in charcoal-stripped serum and transferred to 96-well microtiter plates one day prior to treatment with various concentrations (10⁻¹² to 10⁻⁵) of test compounds in the absence (for agonists) and presence (for antagonists) of known glucocorticoid receptor agonists (i.e., dexamethasone, hydrocortisone) for up to 24 hours. Treatments are performed in triplicate. Cell lysates are prepared and luciferase activity is determined using a luminometer. Agonist activity is assessed by comparing the luciferase activity from cells treated with test compound to cells treated with the agonist dexamethasone. Antagonist activity is assessed by comparing the luciferase activity of an EC₅₀ concentration of dexamethasone in the absence and presence of test compound. The EC₅₀ (concentration that produced 50% of the maximal response) for dexamethasone is calculated from dose response curves.

The following is a description of an assay for determining the competitive inhibition binding of the Human Type II Glucocorticoid receptor expressed in Sf9 cells:

Binding protocol: Compounds are tested in a binding displacement assay using human glucocorticoid receptor expressed in Sf9 cells with ³H-dexamethasone as the ligand. Human glucocorticoid receptor is expressed in Sf9 cells as described in Mol. Endocrinology 4: 209,1990. Pellets containing Sf9 cells expressing the human GR receptor from 1 L vats are lysed with 40 ul of 20 mM AEBSF stock (Calbiochem, LaJolla, Calif.) containing 50 mg/ml leupeptin and 40 ml of homogenization buffer is added. The assay is carried out in 96-well polypropylene plates in a final volume of 130 ul containing 200 ug Sf9 lysate protein, 6.9 nM ³H-dexamethasone (Amersham, Arlington Heights, Ill.) in presence of test compounds, test compound vehicle (for total counts) or excess dexamethasone (7 uM non-radioactive, to determine non-specific binding) in an appropriate volume of assay buffer. All compounds are tested at 6 concentrations in duplicate (concentration range 0.1-30 nM-or 3-1000 nM). Test compounds are diluted from a 25 mM stock in 100% DMSO with 70%EtOH and added in a volume of 2 μl. Once all additions are made the plates are shaken, sealed with sealing tape and incubated at 4° C. overnight.

After the overnight incubation, unbound counts are removed with dextran coated charcoal as follows: 75 μl of dextran coated charcoal (5.0 g activated charcoal, 0.5 g dextran adjusted to volume of 100 ml with assay buffer) is added, plates are shaken and incubated for five minutes at 4° C. Plates are then centrifuged in a refrigerated benchtop centrifuge at top speed for 15 minutes. 100 μl of the supernatant from each well is placed into a 96-well PET plate with 200 μl of scintillation cocktail and counted on a beta counter (1450 MicroBetaTrilux, from Wallac, Turku, Finland).

Data analysis: After subtracting non-specific binding, counts bound are expressed as % of total counts. The concentration response for test compounds are fitted to a sigmoidal curve to determine the IC₅₀ (concentration of compound that displaces 50% of the bound counts).

Reagents: Assay Buffer: 2.0 ml 1M Tris, 0.2 ml 0.5mM EDTA, 77.1 mg DTT, 0.243 g sodium molybdate in a volume of 100 ml water; Homogenization buffer: 2.0 ml 0.5 M K₂HPO₄ (pH 7.6), 20 μl 0.5 M EDTA (pH 8.0), 77.1 mg DTT, 0.486 g sodium molybdate in a volume of 100 ml water.

The following is a description of an assay for determining receptor selectivity: T47D cells from ATCC containing endogenous human progesterone and mineralocorticoid receptors are transiently transfected with a 3×GRE-luciferase using Lipofectamine Plus (GIBCO-DRL, Gaithersburg, Md.). Twenty-four hours post-transfecton cells are maintained in charcoal-stripped serum and transferred to 96-well microtiter plates. The next day cells are treated with various concentrations (10⁻¹² to 10⁻⁵) of test compounds in the absence and presence of a known progesterone receptor agonist (progesterone) and a known mineralocorticoid receptor agonist (aldosterone) for up to 24 hours. Treatments are performed in triplicate. Cell lysates are prepared and luciferase activity is determined using a luminometer. Agonist activity is assessed by comparing the luciferase activity from cells treated with compound alone to cells treated with either the agonist progesterone or aldosterone. Antagonist activity is assessed by comparing the luciferase activity of an EC₅₀ concentration of progesterone or aldosterone in the absence and presence of compound. The EC₅₀ (concentration that produced 50% of maximal response) for progesterone and aldosterone is calculated from dose response curves. The following is a description of an assay for determining the ability of a compound to inhibit glucocorticoid agonist induction of liver tyrosine amino transferase (TAT) activity in conscious rats:

Animals: Male Sprague Dawley rats (from Charles River, Wilimington Mass.) (adrenal-intact or adrenalectomized at least one week prior to the screen) b.w. 90 g are used. The rats are housed under standard conditions for 7-10d prior to use in the screen.

Experimental protocol: Rats (usually 3 per treatment group) are dosed with test compound, vehicle or positive control (Ru486) either i.p. p.o., s.c. or i.v. (tail vein). The dosing vehicle for the test compounds is typically one of the following: 100% PEG 400, 0.25% methyl cellulose in water, 70% ethanol or 0.1 N HCl and the compounds are tested at doses ranging from 10 to 125 mg/kg. The compounds are dosed in a volume of 1.0 ml/100 g body weight (for p.o.) or 0.1 ml/100 g body weight for other routes of administration. Ten minutes after the administration of the test compound, the rats are injected with dexamethasone (0.03 mg/kg i.p. in a volume of 0.1 ml/100 g) or vehicle. To prepare the dexamethasone dosing solution, dexamethasone (from Sigma, St. Louis, Mo.) is dissolved in 100% ethanol and diluted with water (final: 10% ethanol:90% water, vol:vol). Groups treated with vehicle-vehicle, vehicle-dexamethasone, and Ru486-dexamethasone are included in each screen. The compounds are tested vs. dexamethasone only. Three hours after the injection of dexamethasone the rats are sacrificed by decapitation. A sample of liver (0.3 g) is excised and placed in 2.7 ml of ice-cold buffer and homogenized with a polytron. To obtain cytosol the liver homogenate is centrifuged at 105,000 g for 60 min and the supernatant is stored at −80° C. until analysis. TAT is assayed on 100 ul of a 1:20 dilution of the 105,000 g supernatant using the method of Granner and Tomkins (Methods in Enzymology 17A: 633-637, 1970) and a reaction time of 8-10 minutes. TAT activity is expressed as umol product/min/g liver. Interpretation: Treatment data are analyzed by using analysis of variance (ANOVA) with protected least significant difference (PLSD) post-hoc analysis. Compounds are considered active in this test if the TAT activity in the group pretreated with compound prior to dexamethasone administration is significantly (P<0.05) decreased relative to the TAT activity in the vehicle-dexamethasone treated group.

The following is a description of an assay for determining the effect of a compound on two typical genes that are upregulated during an inflammatory response. This assay, the glucocorticoid inhibition of IL-1 (Interleukin-1) induced MMP-1 (Matrix Metalloproteinase-1) and IL-8 (Interleukin-8) production in human chondrosarcoma cells, is conducted as follows: SW1353 human chondrosarcoma cells (obtained from ATCC) from passage 12 through passage 19 are used in a 96 well format assay. Cells are plated at confluence into 96 well plates in DMEM (Dulbecco's Modified Eagle Medium) with 10% fetal bovine serum and incubated at 37° C., 5% CO₂. After 24 hours, serum containing media is removed and replaced with 200 ul/well DMEM containing 1 mg/L insulin, 2 g/L lactalbumin hydrosylate, and 0.5 mg/L ascorbic acid and returned to incubation at 37° C., 5% CO₂. The following morning, the serum free media is removed and replaced with 150 ul/well fresh serum free media containing +/−20 ng/ml IL-1 beta, +/−5 nM dexamethasone, +/−compound. All conditions are completed in triplicate using only the inner 60 wells of the 96 well plate. Outside surrounding wells of plate contain 200 ul of serum free DMEM. Plates are incubated at 37° C., 5% CO₂. At 24 hours after addition of IL-1, 25 ul of sample from each well is removed under aseptic conditions for IL-8 production analysis. Samples are stored at −20° C. until time of analysis. IL-8 production is assessed using the Quantikine human IL-8 ELISA kit from R&D Systems (D8050) on samples diluted 60-fold in RD5P Calibrator Diluent, following the manufacturer's protocol. The percent of the average IL-1 control is determined for the average of each of the triplicate samples following subtraction of the average signal from untreated cells. IC₅₀'s are determined from log linear plots of the percent of control versus the concentration of inhibitor. At 72 hours after IL-1 addition, the remaining media is removed and stored at −20° C. until time of MMP-1 production analysis. MMP-1 production is assessed via the Bio-Trak MMP-1 ELISA kit from Amersham (RPN2610) on 100 ul of neat sample following the manufacturer's protocol.

The percent of the average IL-1 control is determined for the average of each of the triplicate samples following subtraction of the average signal from untreated cells. IC₅₀'s are determined from log linear plots of the percent of control versus the concentration of inhibitor. Dexamethasone has proven to be a good positive control inhibitor of both IL-8 and MMP1 expression (IC₅₀=5nM).

Active compounds are defined as those compounds with: 1) an ED₅₀ of less than 3 μM in the SW 1353 chondrosarcoma GRE luciferase assay; 2) comparatively less than 50% of the maximal activation of dexamethasone at 100 nM in the SW 1353 chondrosarcoma GRE luciferase assay; 3) an average IC₅₀ of less than 3 μM in the IL-8 and MMP-13 production assays; or 4) comparatively greater than 50% of the maximal inhibition of dexamethasone at 100 nM in the IL-8 and MMP-13 production assays.

More preferred active compounds are defined as those compounds with: 1) an ED₅₀ of less than 3 μM in the SW 1353 chondrosarcoma GRE luciferase assay; 2) comparatively less than 40% of the maximal activation of dexamethasone at 100 nM in the SW 1353 chondrosarcoma GRE luciferase assay; 3) an average IC₅₀ of less than 3 μM in the IL-8 and MMP-13 production assays; or 4) comparatively greater than 60% of the maximal inhibition of dexamethasone at 100 nM in the IL-8 and MMP-13 production assays.

The following examples will serve to further typify the nature of this invention but should not be construed as a limitation in the scope thereof, which scope is defined solely by the appended claims.

### EXAMPLE 1

### 4b-Ethyl-7-hydroxy-7-trifluoromethyl-4b,5,6,7,8,8a,9,10-octahydro-phenanthrene-2-carboxylic acid N′-pyridin-2-yl-hydrazide

(4aR, 10aR)-4a-ethyl-7-hydroxy-3,4,4a,9,10,10a-hexahydro-1H-phenanthrene-2-one, a ketone, was used as the starting material for the preparation of the title compound. (4aR, 10aR)-4a-ethyl-7-hydroxy-3,4,4a,9,10,10a-hexahydro-1H-phenanthrene-2-one was prepared by the following procedures:

(a). 1-Ethyl-6-methoxy-3,4-dihydro-1 H-naphthalen-2-one was prepared by heating a solution of 6-methoxy-2-tetralone (120.55 grams, 0.684 mol) and pyrrolidine (61 mL, 0.685 mol) in toluene (1.7 L) was heated to reflux using a Dean-Stark trap apparatus for 3 hours. After removal of the azeotroped water, the reaction mixture was cooled to room temperature and concentrated to a solid. To this solid was added methanol (1.2 L) and ethyl iodide (121 mL, 1.51 mol). The resulting solution was heated at relux overnight and then concentrated under vacuum to remove methanol. A solution of acetic acid (120 mL), sodium acetate (120 g) in water (240 mL) was added to the residue and the resultant mixture was heated at reflux for 2 hours. After cooling, the mixture was extracted several times with diethyl ether. The combined organic layers were washed twice with aqueous 1M HCl, twice with aqueous 1M NaOH and once with brine. After drying over magnesium sulfate, the solvent was evaporated to afford the title compound as an oil, 121.8 grams. Mass spectrum: m/e 204.

(b). A solution of 1-Ethyl-6-methoxy-3,4-dihydro-1H-naphthalen-2-one (121.8 grams, 0.592 mol) and freshly distilled (S)—(−)-alpha-methyl benzylamine (72 grams, 0.592 mol) in toluene (600 mL) was heated at reflux using a Dean-Stark trap apparatus ovemight. After removal of the azeotroped water, some of the toluene (about 300 mL) was distilled off. Freshly distilled methylvinylketone (4.39 grams, 0.626 mol) was added drop wise to the solution. The solution was stirred at room temperature for 2 hours and then heated in an oil bath at 45° C. overnight. The reaction solution was cooled in an ice bath and aqueous 10% sulfuric acid was added. After stirring at room temperature for 2 days, the solution was extracted three times with ethyl acetate (EtOAc). The combined organic layers were washed with water and brine. After drying over magnesium sulfate, the solvent was evaporated and (1S, 9S)-Ethyl-10-hydroxy-5-methoxy-10-methyl-tricyclo[7.3.1.0^{2,7}]trideca-2,4,6-trien-13-one was isolated by flash chromatography eluting with 15% ethyl acetate in hexane followed by 21% ethyl acetate in hexane. Mass spectrum: m/e 275 (M+1).

(c). A solution of 59.6 grams (0.217 mol) of (1 S, 9S)-Ethyl-10-hydroxy-5-methoxy-10-methyl-tricyclo[7.3.1.0^{2,7}]trideca-2,4,6-trien-13-one in methanol (300 mL) was added drop wise to 1 M sodium methoxide in methanol (250 mL). The mixture was heated at reflux for 3 hours. After cooling to room temperature, acetic acid was added to give a neutral pH and the mixture was concentrated under vacuum. The residue was dissolved in ethyl acetate and washed sequentially with aqueous saturated NaHCO₃, water and brine. After drying over magnesium sulfate, the solvent was evaporated to afford (4aR)-4a-Ethyl-7-methoxy-4,4a,9,10-tetrahydro-3H-phenanthrene-2-one as a tan solid, 55 grams. Mass spectrum: 257 (M+1).

(d). To a well stirred solution of (4aR)-4a-Ethyl-7-methoxy-4,4a,9,10-tetrahydro-3H-phenanthrene-2-one (55 grams, 0.214 mol) in methanesulfonic acid (890 mL) was added in portions D,L-methionine (106.7 grams, 0.715 mol). The mixture was stirred overnight at room temperature, then poured into excess ice and stirred for an additional 30 minutes. The precipitated solid was collected by filtration and subsequently dissolved in ethyl acetate. The resultant solution was washed with aqueous saturated sodium bicarbonate (NaHCO₃) and brine. After drying over magnesium sulfate, the solvent was evaporated under vacuum to afford a red semi-solid. This semi-solid was triturated with diethyl ether to afford (4aR)-4a-Ethyl-7-hydroxy-4,4a,9,10-tetrahydro-3H-phenanthren-2-one (Compound 1) as a yellow solid (34 grams), which was collected by filtration. ¹H NMR (CDCl₃) δ 7.14 (d, J=8.3 Hz, 1 H), 6.76 (dd, J=2.6, 8.3 Hz, 1 H), 6.62 (d, J=2.6 Hz, 1 H), 5.97 (s, 1H), 3.00-2.95 (m, 1 H), 2.86-2.38 (series of m, total 6 H), 2.08-1.90 (m, 3 H), 0.84 (t, J=7.3 Hz, 3 H).

The title compound in this example was then prepared by the following procedures:

(a). Preparation of 4a-Ethyl-2-methyl-1,2,3,4,4a,9,10,10a-octahydro-phenanthrene-2,7-diol.

(4a-Ethyl-7-hydroxy-3,4,4a,9,10,10a-hexahydro-1 H-phenanthren-2-one (2.066 g, 8.455 mmol) and cesium fluoride were dried under high vacuum by azeotroping off toluene (1 ml). The ketone and cesium fluoride were dissolved in tetrahydrofuran (20 ml), and then trimethyl(trifluoromethyl) silane (47 ml, 23.5 mmol) was added. The reaction solution was stirred for 7 hours at room temperature and then concentrated in vacuo. The residue was dissolved in tetrahydrofuran (20 ml), followed by the addition of 1.0 M tetrabutylammonium fluoride in tetrahydrofuran (20 ml). The reaction solution was then stirred at room temperature overnight, quenched with a few drops of water, and concentrated in vacuo. The residue was partitioned between water and methylene chloride (100 ml). The aqueous layer was further extracted with methylene chloride (100 ml). The combined organic layers were washed with brine, dried over magnesium sulfate and concentrated in vacuo. The product was isolated by flash chromatography on silica gel eluting with 4:1 of hexanes/ethyl acetate to afford 2 in quantitative yield (2.78 g), MS: 314.35.

(b). Preparation of methanesulfonic acid 4b-ethyl-7-hydroxy-7-methyl-4b,5,6,7,8,8a,9,10-octahydro-phenanthren-2-yl ester.

4a-Ethyl-2-methyl-1,2,3,4,4a,9,10,10a-octahydro-phenanthrene-2,7-diol (2.78 g, 8.455 mmol) was dissolved in THF (200 ml), followed by the addition of sodium hydride (60% in mineral oil) (406 mg, 10.146 mmol), and the resultant solution was stirred for 30 minutes. N-phenyltrifluoromethane sulfonimide (3.625 g, 10.146 mmol) was added and the reaction mixture was stirred over the weekend at room temperature. The reaction solution was concentrated in vacuo. Water (30 ml) was added to the residue and the product was extracted with diethyl ether (50 ml). The aqueous layer was further extracted with ethyl acetate (3 x 40 ml). The organic layers were combined, dried over magnesium sulfate, and concentrated in vacuo. The triflate product (2.73 g, 72% yield) was obtained after purification by flash chromatography with 4:1 of hexanes/ethyl acetate, MS: 446.41.

(c). Preparation 4b-Ethyl-7-hydroxy-7-methyl4b,5,6,7,8,8a,9,10-octahydro-phenanthrene-2-carboxylic acid methyl ester.

A flask was charged with palladium (II) acetate (105 mg, 0.428 mmol) and bisdiphenylphosphenopropane (177 mg, 0.428 mmol) in dimethylsulfoxide (75 ml). A solution of methanesulfonic acid 4b-ethyl-7-hydroxy-7-methyl-4b,5,6,7,8,8a,9,10-octahydro-phenanthren-2-yl ester (2.73 g, 6.121 mmol) in methanol (75 ml) was added to the flask, followed by triethylamine (2.22 ml, 15.303 mmol). Carbon monoxide was bubbled into the solution for 45 minutes. The flask was then fitted with a carbon monoxide-filled balloon and heated at 90° C. overnight. The reaction was cooled to room temperature, and the flask was purged with nitrogen. The reaction mixture was concentrated in vacuo to remove methanol. Water (100 ml) was added, and the aqueous solution was extracted with ethyl acetate (3×100 ml). The combined organic layers were washed with brine, dried over magnesium sulfate, and concentrated in vacuo. The product (4) (1.778 g, 82% yield) was obtained after purification by flash chromatography eluting with hexanes/ethyl acetate 4:1, MS: 356.39.

(d). Preparation of 4b-Ethyl-7-hydroxy-7-methyl-4b,5,6,7,8,8a,9,10-octahydro-phenanthrene-2-carboxylic acid

4b-Ethyl-7-hydroxy-7-methyl-4b,5,6,7,8,8a,9,10-octahydro-phenanthrene-2-carboxylic acid methyl ester (60 mg, 0.168 mmol) was combined with lithium hydroxide monohydrate (35 mg, 0.842 mmol) in methanol (8 ml) and water (1 ml). The reaction mixture was heated at 60° C. over the weekend. TLC with hexanes/ethyl acetate 2:1 showed complete conversion of starting material. The reaction mixture was concentrated in vacuo. Water (2 ml) was added to the residue, and the pH was adjusted to 2 with the addition of 1 N hydrochloric acid. The aqueous mixture was extracted with ethyl acetate (2×100 ml). The combined organic layers were washed with brine, dried over magnesium sulfate, and concentrated in vacuo. The product was obtained in quantitative yield, MS: 342.36.

(e). Preparation of 4b-Ethyl-7-hydroxy-7-trifluoromethyl-4b,5,6,7,8,8a,9,10-octahydro-phenanthrene-2-carboxylic acid N′-pyridin-2-yl-hydrazide

To the solution of 4b-Ethyl-7-hydroxy-7-methyl-4b,5,6,7,8,8a,9,10-octahydro-phenanthrene-2-carboxylic acid (32 mg, 0.0935 mmol), 2-hydrazinopyridine (12 mg, 0.112 mmol), EDC (22 mg, 0.112 mmol), and 1-hydroxybenzotriazole (15 mg, 0.112 mmol) was added methylene chloride (3 ml) to form a suspension. Diisopropylethylamine (39 μl, 0.224 mmol) was added, and the reaction mixture was turned to a clear solution. The reaction solution was stirred at room temperature for 3 days under nitrogen. The reaction mixture was loaded on a prep-TLC plate and eluted with 1:29 of 7 M ammonia in methanol/methylene chloride to afford the title compound, yield: 44 mg; MS: 433.48.

### EXAMPLE 2

### Preparation of 4b-Benzyl-7-hydroxy-7-trifluoromethyl-4b,5,6,7,8,8a,9,10-octahydro-phenanthrene-2-carboxylic acid N′-pyridin-2-yl-hydrazide

The synthesis of 4b-Benzyl-7-hydroxy-7-methyl-4b,5,6,7,8,8a,9,10-octahydro-phenanthrene-2-carboxylic acid was described in U.S. Pat. No. 6,380,223B1 and EP1201655A2 and the content of which is incorporated herein by reference.

The title compound of this example, 4b-Benzyl-7-hydroxy-7-trifluoromethyl-4b,5,6,7,8,8a,9,10-octahydro-phenanthrene-2-carboxylic acid N′-pyridin-2-yl-hydrazide (8) was prepared by the following procedures:

To the solution of 4b-Benzyl-7-hydroxy-7-methyl-4b,5,6,7,8,8a,9,10-octahydro-phenanthrene-2-carboxylic acid (1.000 g, 2.475 mmol), 2-hydrazinopyridine (324 mg, 2.970 mmol), EDC (569 mg, 2.970 mmol), and 1-hydroxybenzotriazole (401 mg, 2.970 mmol) was added methylene chloride (60 ml) to form a suspension. Diisopropylethylamine (1.03 ml, 5.940 mmol) was added, and the reaction mixture was turned to a clear solution. The reaction solution was stirred at room temperature for 24 hours under nitrogen. The organic reaction mixture was diluted with CH₂Cl₂, washed with saturated ammonium chloride (2×60 ml) and brine (60 ml). The organic layer was dried over magnesium sulfate and concentrated in vacuo. The crude product was purified by flash chromatography using 1:19 of 7 M ammonia in methanol/methylene chloride to afford 8, yield: 880 mg; MS: 495.55.

### EXAMPLE 3

### Preparation of 4b-Ethyl-6,7-dihydroxy-6-methyl-7-thiazol-2-yl-4b,5,6,7,8,8a,9,10-octahydro-phenanthrene-2-carboxylic acid N′-pyridin-2-yl-hydrazide

The preparation of 4b-Ethyl-6,7-dihydroxy-6-methyl-7-thiazol-2-yl-4b,5,6,7,8,8a,9,10-octahydro-phenanthrene-2-carboxylic acid N′-pyridin-2-yl-hydrazide involves the following four steps (a), (b), (c) and (d):

(a). Preparation of (2R, 3R, 4aR, 10aR)4a-Ethyl-3-methyl-2-thiazol-2-yl-1,2,3,4,4a,9,10,10a-octahydrophenanthrene-2,3,7-triol.

A solution of 2-bromothiazole (0.27 mL, 2.9 mmol) in tetrahydrofuran (10 mL) was cooled to −78° C. and treated with a 2.5 M solution of n-butyllithium in hexane (1.1 mL, 2.75 mmol) to give a dark solution. A solution of the compound of Preparation 11 f (75 mg, 0.193 mmol) in tetrahydrofuran was then added via canula. The mixture was stirred at −78° C. for 3 hours and then quenched with aqueous saturated ammonium chloride solution. After the mixture was diluted with a little water and warmed to room temperature, it was extracted five times with ethyl acetate. The combined organic layers were washed with brine, dried over magnesium sulfate and concentrated. The residue taken up in tetrahydrofuran (5 mL), treated with a 1 M solution of tetrabutylammonium fluoride in tetrahydrofuran (0.39 mL, 0.39 mmol) and stirred at room temperature overnight. The reaction mixture was filtered through a pad of Celite® and concentrated. The title compound was purified by preparative HPLC. Mass spectrum (m/e) 360 (M⁺+1).

(b). Preparation of methanesulfonic acid 4b-ethyl-6,7-dihydroxy-6-methyl-7-thiazol-2-yl4b,5,6,7,8,8a,9,10-octahydro-phenanthren-2-yl ester

(2R, 3R, 4aR, 10aR)4a-Ethyl-3-methyl-2-thiazol-2-yl-1,2,3,4,4a,9,10,10a-octahydrophenanthrene-2,3,7-triol (0.986 g, 2.7429 mmol) was dissolved in THF (100 ml) in a flame-dried flask, followed by the addition of sodium hydride (60% in mineral oil) (76 mg, 3.0171 mmol) and N-phenyltrifluoromethane sulfonimide (1.08 g, 3.0171 mmol). The reaction solution was allowed to stir overnight at room temperature. The reaction was quenched with water (30 ml) and diluted with diethyl ether (75 ml). The product was extracted with ethyl acetate (3×30 ml), dried over magnesium sulfate, and concentrated in vacuo to afford a yellow oil which was purified by flash chromatography on silica gel using a gradient of hexanes/ethyl acetate to generate methanesulfonic acid 4b-ethyl-6,7-dihydroxy-6-methyl-7-thiazol-2-yl-4b,5,6,7,8,8a,9,10-octahydro-phenanthren-2-yl ester, yield: 1.27 g.

(c). Preparation of 4b-Ethyl-6,7-dihydroxy-6-methyl-7-thiazol-2-yl-4b,5,6,7,8,8a,9,10-octahydro-phenanthrene-2-carboxylic acid methyl ester

A flame-dried flask was charged with palladium (II) acetate (40.7 mg, 0.1811 mmol) and bisdiphenylphosphenopropane (74.7 mg, 0.1811 mmol) in dimethylsulfoxide (50 ml). A solution of methanesulfonic acid 4b-ethyl-6,7-dihydroxy-6-methyl-7-thiazol-2-yl4b,5,6,7,8,8a,9,10-octahydro-phenanthren-2-yl ester (1.272 g, 2.5878 mmol) in methanol (50 ml) was added to the flask, followed by the addition of triethylamine (0.94 ml, 6.4695 mmol). Carbon monoxide was bubbled into the solution for 25 minutes. The flask was then fitted with a carbon monoxide-filled balloon and heated at 90° C. for 18 hours. The reaction was quenched with water (75 ml), and the product was extracted with ethyl acetate (5×50 ml). The combined organic layers were washed with brine (25 ml), dried over sodium sulfate, filtered through celite, and concentrated to dryness. The crude product was purified by flash chromatography on silica gel using a gradient of hexanes/ethyl acetate to afford 4b-Ethyl-6,7-dihydroxy-6-methyl-7-thiazol-2-yl-4b,5,6,7,8,8a,9,10-octahydro-phenanthrene2-carboxylic acid methyl ester, yield: 0.844 g.

(d). Preparation of 4b-Ethyl-6,7-dihydroxy-6-methyl-7-thiazol-2-yl-4b,5,6,7,8,8a,9,10-octahydro-phenanthrene-2-carboxylic acid

4b-Ethyl-6,7-dihydroxy-6-methyl-7-thiazol-2-yl-4b,5,6,7,8,8a,9,10-octahydro-phenanthrene-2-carboxylic acid methyl ester (0.844 g, 2.102 mmol) was combined with lithium hydroxide monohydrate (0.44; g, 10.51 mmol) in methanol (75 ml) and water (10 ml). The reaction mixture was heated at 60° C. for 3 hours. A few drops of hydrochloric acid were added to acidify the reaction mixture, and then the reaction mixture was concentrated in vacuo to remove methanol. The aqueous layer was diluted with 1.0 M sodium hydroxide (75 ml) and washed with diethyl ether (20 ml). The aqueous layer was then re-acidified to pH 3, and the product was extracted with ethyl acetate. The organic layer was dried with magnesium sulfate and concentrated in vacuo to afford 4b-Ethyl-6,7-dihydroxy-6-methyl-7-thiazol-2-yl-4b,5,6,7,8,8a,9,10-octahydro-phenanthrene-2-carboxylic acid, yield: 0.483 g.

(e). Preparation of 4b-Ethyl-6,7-dihydroxy-6-methyl-7-thiazol-2-yl-4b,5,6,7,8,8a,9,10-octahydro-phenanthrene-2-carboxylic acid N′-pyridin-2-yl-hydrazide

4b-Ethyl-6,7-dihydroxy-6-methyl-7-thiazol-2-yl-4b,5,6,7,8,8a,9,10-octahydro-phenanthrene-2-carboxylic acid (31 mg, 0.08 mmol) was combined with 2-Hydrazinopyridine (13 mg, 0.12 mmol), EDC (18 mg, 0.096 mmol), and 1-Hydroxybenzotriazole (13 mg, 0.010 mmol) in methylene chloride (5 ml). The reaction mixture was stirred overnight at room temperature. The reaction mixture was dissolved in a minimal amount of dimethylformamide and purified by flash chromatography on silica gel using ethyl acetate/hexanes (1:1) to afford 4b-Ethyl-6,7-dihydroxy-6-methyl-7-thiazol-2-yl-4b,5,6,7,8,8a,9,10-octahydro-phenanthrene-2-carboxylic acid N′-pyridin-2-yl-hydrazide, yield: 15 mg.

The activity of the compounds of the present invention are demonstrated by one or more of the assays described in U.S. Patent No. 6,380,223, the content of which is incorporated herein by reference. These assays include (1) an assay for the identification of GR antagonists/agonist, (2) an assay for determining the competitive inhibition binding of the Human Type II GR expressed in Sf9 cells, (3) an assay for determining receptor selectivity: T47D cells from ATCC containing endogenous human progesterone and mineralocorticoid receptors, (4) an assay for determining anti-diabetes and anti-obesity activity and (5) an assay for determining the ability of a compound to inhibit glucocorticoid agonist induction of liver tyrosine amino transferase (TAT) activity in conscious rats.

### EXAMPLE 4

### Gelatin Capsules

Hard gelatin capsules are prepared using the following:

| Ingredient | Quantity (mg/capsule) |
|---|---|
| Active ingredient | 0.25-100 |
| Starch, NF | 0-650 |
| Starch flowable powder | 0-50 |
| Silicone fluid 350 centistokes | 0-15 |

### EXAMPLE 5

### Tablet Formulation

| Ingredient | Quantity (mg/tablet) |
|---|---|
| Active ingredient | 0.25-100 |
| Cellulose, microcrystalline | 200-650 |
| Silicon dioxide, fumed | 10-650 |
| Stearic acid | 5-15 |

The components are blended and compressed to form tablets.

### EXAMPLE 6

### Suspensions

| Ingredient | Quantity (mg/5 ml) | |
|---|---|---|
| Active ingredient | 0.25-100 | mg |
| Sodium carboxymethyl cellulose | 50 | mg |
| Syrup | 1.25 | mg |
| Benzoic acid solution | 0.10 | ml |
| Flavor | q.v. | |
| Color | q.v. | |
| Purified Water to | 5 | mL |

The active ingredient is passed through a No. 45 mesh U.S. sieve and mixed with the sodium carboxymethyl cellulose and syrup to form smooth paste. The benzoic acid solution, flavor, and color are diluted with some of the water and added, with strring. Sufficient water is then added to produce the required volume. An aerosol solution is prepared containing the following ingredients:

1. A compound of the formula I 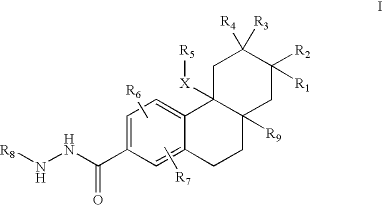
an isomer thereof, a prodrug of said compound or isomer, or a pharmaceutically acceptable salt of said compound, isomer or prodrug;
wherein R₁ is a) —H, b) —(C₁-C₆)alkyl-A—(C₁-C₆)alkyl, or —(C₁-C₃)alkyl-A—(C₁-C₃)alkyl-A-(C₀-C₃)alkyl, wherein A for each occurrence is independently S, O, N, OH or NH₂; wherein each carbon atom is optionally substituted with 1 or 2 Rₓ, c) —(C₂-C₁₀)alkenyl optionally substituted with 1 or 2 Rₓ, d) —(C₂-C₁₀)alkynyl, -ethynyl (C₁-C₈)alkoxy or —(C₁-C₄)alkoxy(C₁-C₄)alkylethynyl, wherein each carbon atom is optionally substituted with 0, 1 or 2 Rₓ, e) —CH═C═CH₂, f) —CN, g) —(C₃-C₉)cycloalkyl, h) —Z—(C₆-C₁₀)aryl, i) —Z-het, j) —C(O)O(C₁-C₆)alkyl, k) —O(C₁-C₆)alkyl, l) —Z—S—R₁₂, m) —Z—S(O)—R₁₂, n) —Z—S(O)₂—R₁₂, o) —(C₁-C₈)alkyl, wherein each carbon atom is optionally substituted with 1, 2, or 3 halo, p) —NR₁₂O—(C₁-C₆)alkyl or q) —CH₂ORₓ;
Z for each occurrence is independently a) —(C₀-C₆)alkyl, b) —(C₂-C₆)alkenyl or c) —(C₂-C₆)alkynyl;
Rₓ for each occurrence is independently a) —OH, b) -halo, c) —Z—(C₁-C₈)alkyl, wherein each carbon atom is optionally substituted with 1, 2, or 3 halo, d) —CN, e) —NR₁₂R₁₃, f) —(C₃-C₆)cycloalkyl, g) —(C₃-C₆)cycloalkenyl, h) —(C₀-C₃)alkyl-(C₆-C₁₀)aryl, i) -het or j) —N₃;
wherein het is a 5-, 6- or 7-membered saturated, partially saturated or unsaturated ring containing from one to three heteroatoms independently selected from the group consisting of nitrogen, oxygen and sulfur; and including any bicyclic group in which any of the above heterocyclic rings is fused to a benzene ring or another heterocycle; and the nitrogen may be in the oxidized state giving the N-oxide form; and optionally substituted with 1, 2 or 3 R_{y};
R_{y} for each occurrence is independently a) -halo, b) —OH, c) —(C₁-C₆)alkyl, d) —(C₂-C₆)alkenyl, e) —(C₂-C₆)alkynyl, f) —O(C₁-C₆)alkyl, g) —O(C₂-C₆)alkenyl, h) —O(C₂-C₆)alkynyl, i) —(C₀-C₆)alkyl—NR₁₂R₁₃, j) —C(O)—NR₁₂R₁₃, k) —Z—SO₂R₁₂, l)—Z—SOR₁₂, m) —Z-SR₁₂, n) —NR]₂—SO₂R₁₃, o) —NR₁₂—C(O)—R₁₃, p) —NR₁₂—OR₁₃, q) —SO₂—NR₁₂R₁₃, r) —CN, s) —CF₃, t) —C(O)(C₁-C₆)alkyl, u) ═O, or v) —Z—SO₂-phenyl;
R₂, R₃ and R₄ are each independently a) —H, b) -halo, c) —OH, d) —(C₁-C₁₀)alkyl, wherein each carbon atom is optionally substituted with 1, 2 or 3 Rₓ, e) —NR₁₂R₁₃, f) —Z—C(O)O(C₁-C₆)alkyl, g) —Z—C(O)NR₁₂R₁₃, h) (C₁-C₆)alkoxy, i) —Z—O-C(O)—(C₁-C₆)alkyl, j) —Z—O—(C₁-C₃)alkyl-C(O)—NR₁₂R₁₃, k) —Z—O—(C₁-C₃)alkyl-C(O)—O(C₁-C₆)alkyl, l) —O—(C₂-C₆)alkenyl, m) —O—(C₂-C₆)alkynyl, n) —O—Z-het, o) —COOH, p) —C(OH)R₁₂R₁₃ or q) —Z—CN;
R₁₂ and R₁₃ for each occurrence are each independently a) —H, b) —(C₁-C₆)alkyl wherein 1 or 2 carbon atoms, other than the connecting carbon atom, may optionally be replaced with 1 or 2 heteroatoms independently selected from S, 0 and N and wherein each carbon atom is optionally substituted with 1, 2 or 3 halo, c) —(C₂-C₆)alkenyl optionally substituted with 1, 2 or 3 halo or d) —(C₂-C₆)alkynyl wherein 1 carbon atom, other than the connecting carbon atom and the ethynyl atoms, may optionally be replaced with 1 oxygen atom and wherein each carbon atom is optionally substitute with 1, 2 or 3 halo;
or R₁₂ and R₁₃ are taken together with N to which they are attached to form het;
X is a) absent, b) —CH₂—, c) —CH(OH)— or d) —C(O)—;
R₅ is a) —H, b) —Z—CF₃, c) —(C₁-C₆)alkyl, d) —(C₂-C₆)alkenyl, e) —(C₂-C₆)alkynyl, f) —(C₆-C₁₀)aryl, g) —CHO, h) —CH=N—OR₁₂, i) —Z—C(O)OR₁₂, j) —Z—C(O)—NR₁₂R₁₃, k) —Z—C(O)—NR₁₂—Z-het, l) —Z—NR₁₂R₁₃, m) —Z—NR₁₂het, n) —Z-het, o) —Z—O-het, p) —Z—(C₆-C₁₀)aryl, q) —Z—O—(C₆-C₁₀)aryl, r) —CHOH—(C₆-C₁₀)aryl or s) —C(O)—(C₆-C₁₀)aryl wherein said (C₆-C₁₀)aryl is optionally substituted with 1 or 2 of the following: —Z—OH, —Z—NR₁₂R₁₃, —Z—NR₁₂-het, —C(O)NR₁₂R₁₃, —C(O)O(C₁-C₆)alkyl, —C(O)OH, —C(O)-het, —NR₁₂—C(O)—(C₁-C₆)alkyl, —NR₁₂—C(O)—(C₂-C₆)alkenyl, —NR₁₂—C(O)—(C₂-C₆)alkynyl, —NR₁₂—C(O)—Z-het, —CN, —Z-het, —O—(C₁-C₃)alkyl-C(O)—NR₁₂R₁₃, —O—(C₁-C₃)alkyl-C(O)O(C₁-C₆)alkyl, —NR₁₂—Z—C(O)O(C₁-C₆)alkyl, —N(Z—C(O)O(C₁-C₆)alkyl)₂, —NR₁₂—Z—C(O)—NR₁₂R₁₃, —Z—NR₁₂—SO₂—R₁₃, —NR₁₂—SO₂-het, —C(O)H, —Z—NR₁₂—Z—O(C₁-C₆)alkyl, —Z—NR₁₂—Z—NR₁₂R₁₃, —Z—NR₁₂—(C₃-C₆)cycloalkyl, —Z—N(Z—O(C₁-C₆)alkyl)₂, —SO₂R₁₂, —SOR₁₂, —SR₁₂, —SO₂NR₁₂R₁₃, —O—C(O)—(C₁-C₄)alkyl, —O—SO₂—(C₁-C₄)alkyl, -halo or —CF₃;
R₆ and R₉ are each independently a) —H, b) -halo, c) (C₁-C₆)alkyl substituted with 0 to 3 halo, d) —(C₂-C₆)alkenyl substituted with 0 to 3 halo, e) —(C₂-C₆)alkynyl optionally substituted with 1, 2 or 3 halo, f) —CN, g) —(C₃-C₆)cycloalkyl, h) —(C₃-C₆)cycloalkenyl, i) —OH, j) —O—(C₁-C₆)alkyl, k) —O—(C₁-C₆)alkenyl, l) —O—(C₁-C₆)alkynyl, m) —NR₁₂R₁₃, n) —C(O)OR₁₂ or o) —C(O)NR₁₂R₁₃;
R₇ is a) —H, b) —(C₁-C₁₀)alkyl optionally substituted with 1, 2 or 3 substituents independently selected from -halo, —OH and —N₃, c) —(C₂-C₁₀)alkenyl optionally substituted with 1, 2 or 3 substituents independently selected from -halo, —OH and —N₃, d) —(C₂-C₁₀)alkynyl optionally substituted with 1, 2 or 3 substituents independently selected from -halo, —OH and —N₃, e) -halo, f) —Z—CN, g) —OH, h) —Z-het, i) —Z—NR₁₂R₁₃, j) —Z—C(O)-het, k) —Z—C(O)—(C₁-C₆)alkyl, l) —Z—C(O)—NR₁₂R₁₃, m) —Z—C(O)—NR₁₂—Z—CN, n) —Z—C(O)—NR₁₂—Z-het, o) —Z—C(O)—NR₁₂—Z—(C₆-C₁₀)aryl, p) —Z—C(O)—NR₁₂—Z—NR₁₂R₁₃, q) —Z—C(O)—NR₁₂—Z—O(C₁-C₆)alkyl, r) —C₀-C₆)alkyl-C(O)OH, s) —Z—C(O)O(C₁-C₆)alkyl, t) —Z—O—(C₀-C₆)alkyl-het, u) —Z—O—(C₀-C₆)alkyl—(C₆-C₁₀)aryl, v) —Z—O—(C₁-C₆)alkyl optionally substituted with 1 or 2 R_{y}, w) —Z—O—(C₁-C₆)alkyl-CH(O), x) —Z—O—(C₁-C₆)alkyl—NR₁₂-het, y) —Z—O—Z-het-Z-het, z) —Z—O—Z-het-Z—NR₁₂R₁₃, a1) —Z—O—Z-het-C(O)-het, b1) —Z—O—Z—C(O)-het, c1) —Z—O—Z—C(O)-het-het, d1) —Z—O—Z—C(O)—(C₁-C₆)alkyl, e1) —Z—O—Z—C(S)—NR₁₂R₁₃, f1) —Z—O—Z—C(O)—NR₁₂R₁₃, g1) —Z—O—Z—(C₁-C₃)alkyl-C(O)—NR₁₂R₁₃, h1) —Z—O—Z—C(O)—O(C₁-C₆)alkyl, i1) —Z—O—Z—C(O)—OH, j1) —Z—O—Z—C(O)—NR₁₂—O(C₁-C₆)alkyl, k1) —Z—O—Z—C(O)—NR₁₂—OH, l1) —Z—O—Z—C(O)—NR₁₂—Z—NR₁₂R₁₃, m1) —Z—O—Z—C(O)—NR₁₂—Z-het, n1) —Z—O—Z—C(O)—NR₁₂—SO₂—(C₁-C₆)alkyl, ol) —Z—O—Z—C(═NR₁₂)(NR₁₂R₁₃), p1) —Z—O—Z—C(═NOR₁₂)(NR₁₂R₁₃), q1) —Z—NR₁₂—C(O)—O—Z—NR₁₂R₁₃, r1) —Z—S—C(O)—NR₁₂R₁₃, s1) —Z—O—SO₂—(C₁-C₆)alkyl, t1) —Z—O—SO₂—(C₆-C₁₀)aryl, u1) —Z—O—SO₂—NR₁₂R₁₃, v1) —Z—O—SO₂—CF₃, w1) —Z—NR₁₂C(O)OR₁₃ or x1) —Z—NR₁₂C(O)R₁₃;
R₈ is het.

2. The compound of claim 1, wherein het in all instances is a heteroaryl having five to seven members.

3. The compound of claim 1, wherein R₁ is a) —H, b) —(C₁-C₁₀)alkyl, wherein each carbon atom is optionally substituted with 1, 2 or 3 Rₓ, c) —(C₂-C₁₀)alkenyl optionally substituted with 1 or 2 Rₓ, d) —(C₂-C₁₀)alkynyl, wherein each carbon atom is optionally substituted with 1 or 2 Rₓ, e) —(C₃-C₆)cycloalkyl, f) —Z—(C₆-C₁₀)aryl, or g) —Z-heteroaryl having five to seven members;
wherein Rₓ for each occurrence is independently —OH, -halo, and —Z—CF₃;
wherein R₂ is a) —H, b) -halo, c) —OH, d) —(C₁-C₆)alkyl optionally substituted with —OH, e) —Z-heteroaryl having five to seven members, f) —COOH, g) —(C₁-C₁₀)alkyl, wherein each carbon atom is optionally substituted with 1, 2 or 3 Rₓ.

4. The compound of claim 1, wherein R₃ and R₄ are each independently a) —H, b) -halo, c) —OH, d) —(C₁-C₆)alkyl optionally substituted with —OH, e) —Z-heteroaryl having five to seven members, f) —COOH, g) —(C₁-C₁₀)alkyl, wherein each carbon atom is optionally substituted with 1, 2 or 3 Rₓ;
wherein Rₓ for each occurrence is independently —OH, -halo, and —Z—CF₃.

5. The compound of claim 1, wherein R₅ is a) —H, b) —Z—CF₃, c) —(C₁-C₆)alkyl, d) —(C₂-C₆)alkenyl, e) —(C₂-C₆)alkynyl, f) —(C₆-C₁₀)aryl, g) —CHO, h) —CH═N—OR₁₂, i) —Z—C(O)OR₁₂, j) —Z—C(O)—NR₁₂R₁₃, k) —Z—C(O)—NR₁₂—Z-heteroaryl having five to seven members, I) —Z—NR₁₂R₁₃, m) —Z—NR₁₂-heteroaryl having five to seven members, n) —Z-heteroaryl having five to seven members, o) —Z—O-heteroaryl having five to seven members.

6. The compound of claim 1, wherein R₆ and R₉ are each independently a) —H, b) -halo, c) (C₁-C₆)alkyl optionally substituted with 1, 2 or 3 halo, d) —(C₂-C₆)alkenyl optionally substituted with 1, 2 or 3 halo, e) —(C₂-C₆)alkynyl optionally substituted with 1, 2 or 3 halo, f) —CN, g) —(C₃-C₆)cycloalkyl, h) —(C₃-C₆)cycloalkenyl, i) —OH, j) —O—(C₁-C₆)alkyl, k) —O—(C₁-C₆)alkenyl, l) —O—(C₁-C₆)alkynyl, m) —NR₁₂R₁₃, n) —C(O)OR₁₂ or o) —C(O)NR₁₂R₁₃.

7. The compound of claim 1, wherein R₇ is a) —H, b) —(C₁-C₁₀)alkyl optionally substituted with 1, 2 or 3 substituents independently selected from -halo, —OH and —N₃, c) —(C₂-C₁₀)alkenyl optionally substituted with 1, 2 or 3 substituents independently selected from -halo, —OH and —N₃, d) —(C₂-C₁₀)alkynyl optionally substituted with 1, 2 or 3 substituents independently selected from -halo, —OH and —N₃, e) -halo, f) —Z—CN, g) —OH, or h) —Z-heteroaryl having five to seven members.

8. The compound of claim 7, wherein R₈ is a 6-membered unsaturated ring.

9. The compound of claim 1 selected from the group consisting of 4b-Ethyl-7-hydroxy-7-trifluoromethyl4b,5,6,7,8,8a,9,10-octahydro-phenanthrene-2-carboxylic acid N′-pyridin-2-yl-hydrazide, 4b-Benzyl-7-hydroxy-7-trifluoromethyl4b,5,6,7,8,8a,9,10-octahydro-phenanthrene-2-carboxylic acid N′-pyridin-2-yl-hydrazide, 4b-Ethyl-6,7-dihydroxy-6-methyl-7-thiazol-2-yl-4b,5,6,7,8,8a,9,10-octahydro-phenanthrene-2-carboxylic acid N′-pyridin-2-yl-hydrazide.

10. The compound of claim 8, having the formulas III, IV or V: 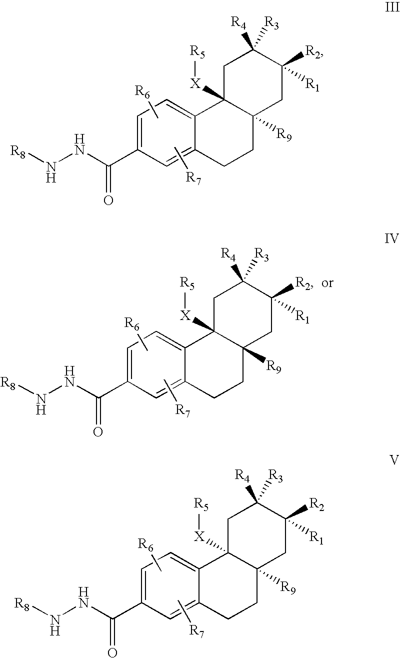
an isomer thereof, a prodrug of said compound or isomer, or a pharmaceutically acceptable salt of said compound, isomer or prodrug;
wherein R₁ is (C₁-C₁₀)alkyl wherein each carbon atom is optionally substituted with 1, 2 or 3 halo or —Z-heteroaryl having five to seven members;
Z is (C₀-C₆)alkyl;
R₂, R₃ and R₄ are each independently a) —H, b) -halo, c) —OH, d) —(C₁-C₁₀)alkyl, wherein each carbon atom is optionally substituted with 1, 2 or 3 —OH, -halo or —Z—CF₃; wherein R₁ is different from R₂ and R₃ is different from R₄;
X is a) absent, or b) —CH₂—;
R₅ is a) —H, b) —Z—CF₃, c) —(C₁-C₆)alkyl, d) —(C₆-C₁₀)aryl or e) —Z-heteroaryl having five to seven members;
R₆ is a) —H, b) -halo, c) (C₁-C₆)alkyl optionally substituted with 1, 2 or 3 halo;
R₇ is —H or —(C₁-C₁₀)alkyl optionally substituted with 1, 2 or 3 substituents independently selected from -halo, —OH and —N₃;
R₈ is a 6-membered unsaturated ring containing from one to three heteroatoms independently selected from the group consisting of nitrogen, oxygen and sulfur;
R₉ is hydrogen.

11. The compound of claim 10, wherein R₃ and R₄ are different.

12. The compound of claim 11 selected from the group consisting of all the isomers of the following compounds: 4b-Ethyl-7-hydroxy-7-trifluoromethyl4b,5,6,7,8,8a,9,10-octahydro-phenanthrene-2-carboxylic acid N′-pyridin-2-yl-hydrazide , 4b-Benzyl-7-hydroxy-7-trifluoromethyl-4b,5,6,7,8,8a,9,10-octahydro-phenanthrene-2-carboxylic acid N′-pyridin-2-yl-hydrazide, 4b-Ethyl-6,7-dihydroxy-6-methyl-7-thiazol-2-yl4b,5,6,7,8,8a,9,10-octahydro-phenanthrene-2-carboxylic acid N′-pyridin-2-yl-hydrazide.

13. A pharmaceutical composition for treating a disorder selected from the group consisting of inflammatory disorders, endocrine disorders; collagen diseases; dermatologic diseases; allergic states; ophthalmic diseases; respiratory diseases; hematologic disorders; neoplastic diseases; edematous states; and gastrointestinal diseases in a mammal comprising (1) the compound of claims 1 or 10, an isomer thereof, a prodrug of said compound or isomer, or a pharmaceutically acceptable salt of said compound, isomer or prodrug and (2) at least one pharmaceutically acceptable carrier, vehicle, diluent, excipient.

14. A method of treating obesity, diabetes, anxiety, or inflammatory diseases in a mammal comprising administering an effective amount of the compound of claim 1, an isomer thereof, a prodrug of said compound or isomer, or a pharmaceutically acceptable salt of said compound, isomer or prodrug.

15. The method of claim 14, wherein said inflammatory disorders are selected from the group consisting of arthritis, asthma, rhinitis and immunomodulation.

16. A pharmaceutical composition comprising (1) the compound of claim 1, (2) a second pharmaceutically active compound, and (3) at least one pharmaceutically acceptable carrier, vehicle, diluent, excipient.

17. The pharmaceutical composition of claim 16, wherein the second pharmaceutically active compound is selected from the group consisting of β₃ agonist, a thyromimetic agent, an eating behavior modifying agent, a NPY antagonist, an aldose reductase inhibitor, a glycogen phosphorylase inhibitor, a sorbitol dehydrogenase inhibitor, insulin, troglitazone, sulfonylureas, glipazide, glyburide, chlorpropamide, a glucocorticoid receptor agonist, a cholinomimetic drug, an anti-Parkinson's drug, an antianxialytic drug, an antidepressant drug, or an antipsychotic drug.

18. A process of preparing compounds of formula I, an isomer thereof, a prodrug of said compound or isomer, or a pharmaceutically acceptable salt of said compound, isomer or prodrug, comprising the step of coupling compound of formula Id with a hydrazine under amide forming conditions: 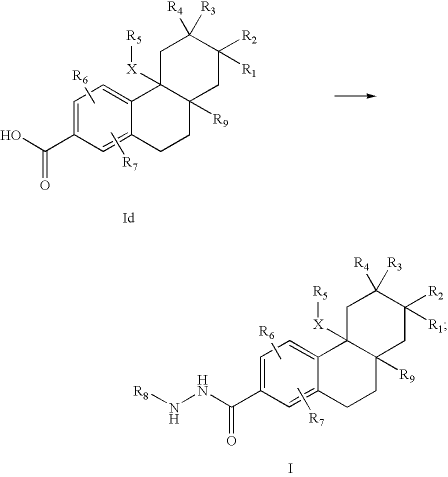
wherein R₁, R₂, R₃, R₄, R₅, R₆, R₇, R₈, R₉ and X are as defined in claim 1.
